(19) United States
(12) Patent Application Publication
(10) Pub. No.: US 2015/0257984 A1
(43) Pub. Date: Sep. 17, 2015

(54) LONG LASTING DENTAL RESTORATIONS AND METHODS FOR PREPARATION THEREOF

(71) Applicants: Ehud SHCHORI, Rehovot (IL); Amir BARAD, Raanana (IL)

(72) Inventors: Ehud SHCHORI, Rehovot (IL); Amir BARAD, Raanana (IL)

(73) Assignee: CORONA DENT LTD., Tel Aviv (IL)

(21) Appl. No.: 14/705,624

(22) Filed: May 6, 2015

Related U.S. Application Data
(62) Division of application No. 13/580,375, filed on Aug. 21, 2012, now abandoned.

(30) Foreign Application Priority Data
Feb. 24, 2010 (IL) 204129

(51) Int. Cl. A61K 6/08

(52) U.S. Cl. CPC A61K 6/08

(57) ABSTRACT

The invention is directed to methods for the preparation of a long-lasting dental restoration, a kit for the preparation of a long-lasting dental restoration comprising one or more containers containing: a first polymerizable compound; a second polymerizable compound; a curing agent for a first polymerization process; a curing agent for a second polymerization process; an inorganic filler; and a pigment; and optionally containing instructions for use thereof. The invention is further directed to a long-lasting dental restoration comprising: a polymeric matrix comprising at least two polymeric systems; an inorganic filler; and a pigment, wherein said long lasting dental restoration is formed by at least two curing processes of said polymeric systems, and to methods for the preparation of a dental restoration, wherein said dental restoration is either fully or partially structured chair-side or it is fully structured in a dental laboratory.

### CROSS-REFERENCE TO RELATED APPLICATIONS

The present divisional patent application claims the benefit of U.S. patent application Ser. No. 13/580,375, filed 21 Aug. 2012, claiming priority from PCT Patent Application no. PCT/IL2011/000175, filed 22 Feb. 2011, claiming priority from Israel Patent Application no. 204129, filed on 24 Feb. 2010.

### FIELD OF THE INVENTION

The present invention relates to dental restorations, particularly long-lasting dental crowns, fillings, bridges, inlays, onlays, and veneers, compositions and methods for preparation thereof.

### BACKGROUND ART

For many years dentistry has enhanced the quality of life of mankind by providing dental restorations for damaged (e.g., broken), decayed, disfigured or missing teeth. Dental restoration can restore both the tooth/teeth's function and appearance. Various dental restorations are employed in dentistry including dental crowns, bridges, inlays/onlays, veneers, fillings, implants, dentures and the like.

In a conventional procedure for preparing a dental restoration, the patient must make multiple visits to the dentist. As commonly practiced today, in the first visit, the dentist examines the tooth that will receive the dental restoration, and may first make an impression of the patient's dental anatomy including the tooth that will ultimately be restored. From such impression, a plaster mold is easily made which faithfully replicates the patient's upper or lower teeth, including the adjacent gum areas. The dentist then prepares the damaged tooth/teeth to receive a dental restoration. First, the dentist may remove any dental caries from the tooth followed by preparation work relevant to the required restoration. Such preparation may involve removing external portions or grinding away external parts, whereby the dimensional size of the tooth is reduced such that the total size of the tooth to be restored is confined within an area less than that of the desired restoration. For example, for covering a damaged tooth with a permanent dental crown, a “crown prep” work is done on the tooth, namely, filing and grinding it to a “core” or “stump”. Next, the dentist takes a final impression of the prepared tooth, from which a second plaster mold is made.

These plaster molds are then sent off to a dental laboratory where a laboratory technician can manufacture a dental restoration. After the restoration is completed it is returned to the dentist who then cements it in place on the patient's prepared tooth/teeth.

Typically when a tooth is prepared to receive a dental restoration it is very visually distractive. To enable a patient to function both physically, namely to masticate food, and to function socially, that is to have a reasonable appearance, the dentist forms a temporary restoration. Temporary crowns, for example, are prepared by molding temporary crown forming material on the prepared tooth and shaping it and hardening it in the patient's mouth so that, hopefully, the temporary crown will last until the permanent one is ready for installation. Various temporary crown and bridge materials are commercially available and/or described in the pertinent art.

At the second office visit, the dentist removes the temporary dental restoration, cleans the tooth, removing any residual temporary cement, and affixes the permanent restoration to the tooth using permanent cement.

This procedure would be satisfactory if it would not take several days up to several weeks from the time the dentist makes the impressions until the dental restoration is returned to the dentist for installation in the patient's mouth. Moreover, temporary crowns and bridges, as well as inlays and onlays, are known to be problematic since they frequently tend to break or dislodge before the patient returns to the dental clinic to receive the permanent restoration.

Thus, the total procedure presently employed for restoring a tooth is time consuming on the part of the dentist and on the part of the patient since it requires the patient to make at least two separate visits to the dental clinic The current procedure wherein the dentist performs all of the dental work in his clinic whereas the permanent restoration itself is manufactured at a remote location not only creates inefficiency but adds to the expenses.

In recent years, new techniques have been developed, particularly relating to dental crowns, whereby a dentist could design and fabricate the crown “chair-side” and mount the crown on the patient's tooth in a single office visit.

For example, U.S. Pat. No. 7,494,339 discloses a method for the preparation of permanent dental restorations, particularly permanent crowns, during a single visit to the dentist's office. The method comprises application of a thin layer of “spacer material” to the upper surface of the prepared tooth. The spacer material is a polymerizing flexible cement composition which serves as a temporary cement that affixes the dental crown in its initial state to the prepared tooth. This flexible cement is preferably made from a catalyst paste and base paste material. A composite crown-forming material dispensed into a substantially transparent plastic matrix is placed in the patient's mouth and molded over the tooth surface; the composite material having a polymerization system capable of being activated by light. Irradiating the composite material in the plastic matrix with light while still in the patient's mouse polymerizes and hardens the material to form a preliminary crown. The preliminary crown is removed from the prepared tooth by breaking the flexible cement, and is substantially hardened by a further curing process to obtain a permanent crown.

The composite crown forming material of U.S. Pat. No. 7,494,339 is made from a catalyst paste and a base paste material. The base paste may comprise a blend of polymerizable compound, polymerization accelerator, and filler material. The polymerizable compound is capable of being hardened to form a polymer network, and is preferably selected from monomers, oligomers, and polymers having one or more ethylenically unsaturated groups such as mono-, di- or poly-acrylates and methacrylates. The catalyst paste comprises a blend of polymerizable compound; polymerization initiator, and filler material. The polymerizable compounds of the base paste are used in the catalyst past as well, provided that the base and catalyst pastes contain at least one different polymerizable compound.

The method described in U.S. Pat. No. 7,494,339 faces few problems: the adjustment of the crown to the prepared tooth and to the neighboring teeth may be problematic since once the crown is hardened it is not possible to add further crown forming material to the inner side of the crown and harden it by curing light. In addition, trimming and polishing the outer surface are difficult since after hardening the crown becomes too hard and crunchy. In addition, the curing light cannot efficiently penetrate into the interproximal areas namely, the areas bordering the neighboring teeth, since these areas are shaded by the neighboring teeth.

U.S. Pat. No. 5,876,209 discloses a method of manufacturing a permanent dental crown which is based on models of the tooth to be crowned made before and after preparation of the restored tooth, which are covered by plastic films, termed “acrylic releasing” foils, that form a septum or a barrier between the models and the crown molded thereon. The acrylic releasing plastic foils, characterized in that they do not adhere to the acrylic material from which the crown is formed, are applied over the models of the intact and prepared tooth by use of heat and pressure. A form is made from the plastic film applied to the model of the intact teeth since it is thicker. Moldable crown forming material is then placed on the plastic foil covered model of the prepared tooth, and the plastic foil form of the intact tooth is placed over it to shape the moldable crown forming material. The crown forming material is partially hardened to create a permanent crown by applying e.g., UV light to it, then subsequently removed from between the foils and further hardened and polished as necessary and cemented onto the patient's prepared tooth. The moldable crown forming material is defined as a polymer-glass moldable ceramic that is commercially available in a plastic state and formulated to remain plastic until subjected to high intensity UV light, which hardens it to a ceramic solid. The method of U.S. Pat. No. 5,876,209 is carried out during a single office visit and eliminates the need to apply a temporary crown to the prepared tooth.

Composite filling material that contains photopolymerizable resins such as an acrylate or methacrylate resin, reinforcing filler particles such as silica and glasses, polymerization initiators, polymerization accelerators, and various additives such as ultraviolet-light absorbers, anti-oxidants, plasticizers, and the like, are known in the art. The composite can be light-cured using a standard dental curing light to form a hardened dental restoration. The curing light normally emits blue visible light in the spectrum of 400 to 500 nm to activate the polymerization initiator. The monomers undergo polymerization and the filling material hardens. In some instances, it is desirable to light-cure the composition in two successive steps. In the first light-curing step, the composition is partially-cured. The dental practitioner can work with the composition shaping and molding it as needed while in the partially-cured state. When ready, the practitioner light-cures the material a second time so that it fully cures and forms a hardened restoration.

U.S. Pat. No. 5,472,991 discloses tooth filling compositions that are curable in two curing steps. The composition comprises polymerizable compounds such as monomeric and polymeric acrylates and methacrylates, filler materials such as silica particles, photoinitiator component I having a light absorption maximum of <450 nm such as bisacylphosphine oxides, and photoinitiator component II having a light absorption maximum of >450 nm such as camphorquinone. The composition is irradiated with light having a wavelength of >450 nm in a partial curing step to form a material having between 50 to 70% of its maximum hardness and then it is irradiated with light at a wavelength of <450 nm so that it completely cures.

US 2008/0220396 discloses methods of making dental restorations such as fillings, inlays, onlays, veneers, crowns, bridges, splints, and dentures using a two-phase light-curing composition. The photopolymerizable composition includes a blend of polymerizable compounds such as monomers and polymers of acrylates and methacrylates; photopolymerization system (a) containing a photoinitiator having a light absorbency maximum wavelength of greater than 420 nm; photopolymerization system (b) containing a photoinitiator having a light absorbency maximum wavelength of less than 420 nm; and a mixture of structural fillers and nanofillers.

There is a need in the dental field for an improved restorative composition having high mechanical strength and pleasing aesthetics. It is highly desirable to develop a method, whereby a dentist could prepare a permanent dental restoration for a patient. Ideally, the dentist would be able to design and fabricate the restoration “chair-side” and mount it on the patient's tooth in a single office visit. It is therefore desirable that the dental restoration forming material will have good working time so that the dentist can handle and place the material more effectively. Thus, it would be highly preferable if the dental forming material composition can be cured in at least two separate and distinct curing steps.

### SUMMARY OF INVENTION

The present invention provides a kit for the preparation of a long-lasting dental restoration. The kit may be used for chair side preparation of a dental restoration by the dentist, or by a dental technician in a dental laboratory. The kit is suitable for the preparation of a long lasting dental restoration selected from a dental crown, a filling, an inlay, an onlay, a veneer, a bridge, a splint or a denture.

The kit comprises one or more containers containing.
(i) a first polymerizable compound;
(ii) a second polymerizable compound;
(iii) a curing agent for a first polymerization process;
(iv) a curing agent for a second polymerization process;
(v) an inorganic filler; and
(vi) a pigment;
   and optionally containing instructions for use thereof.

The kit of the invention preferably further comprises at least one ingredient selected from: an accelerator for a first polymerization process, an accelerator for a second polymerization process, a polymer, a cross-linking agent, a co-reactant, an adhesion promoter such as a silane or titanate derivative, at least one additional polymerizable compound, fibers and microfibers.

When the ingredients of the kit are mixed and the first polymerizable compound is activated by a first curing process, a plastic dental restoration forming material is formed such that it can be shaped and molded, while gradually hardening, to obtain a partially cured dental restoration. This dental restoration is fully cured and hardened to obtain a long-lasting dental restoration when the second polymerizable compound is activated by one or more additional curing processes.

The ingredients of the kit of the invention are designed such that they can form the dental restoration forming material by undergoing curing processes independently selected from a self curing process and a controlled curing process such as a thermo-curing, a photo-curing or a UV-curing process. In certain embodiments, at least one polymerizable compound is curable via a self-curing process, preferably the first polymerizable compound, whereas other polymerizable compounds, preferably the second polymerizable compound, are curable by a controlled curing process.

In certain embodiments, the first polymerization compound is a mono-functional ethylenically unsaturated monomer, preferably a methacrylate derivative, most preferably methyl methacrylate (MMA). In the first polymerization process according to these embodiments, the curing agent and an accelerator form a redox-initiator pair, wherein the curing agent is preferably a peroxide compound, and the accelerator is preferably a tertiary amine.

The second polymerizable compound is selected from a glycidyl ether type epoxy resin, a cycloaliphatic epoxy or epoxidized oil, preferably a glycidyl ether type epoxy resin. The curing agent for the second polymerization system is preferably selected from an anhydride, a carboxylic acid, or a mono- or poly-functional amine

The filler is a powdered natural or synthetic inorganic oxide. In certain embodiments, the filler is a ceramic powder such as a silicate, preferably treated with at least one functional compound such as a silane.

The present invention further provides a long lasting dental restoration selected from a dental crown, a filling, an inlay, an onlay, a veneer, a bridge, a splint or a denture. The long-lasting dental restoration comprises: (i) a polymeric matrix comprising at least two polymeric systems; (ii) an inorganic filler; and (iii) a pigment, and is formed by at least two successive curing processes independently selected from a self curing process and a controlled curing process such as a thermo-curing, a photo-curing or a UV-curing process. The long lasting dental restoration is preferably fabricated chair-said during a single visit to the dentist's office, but may also be made in the dental laboratory.

In certain embodiments, the long lasting dental restoration provided by the present invention is a long-lasting dental crown, most preferably prepared chair-side during a single office visit.

The present invention further provides a method for the preparation of a long-lasting dental restoration comprising a first stage in which a transient dental restoration is formed by application of first curing process to a dental forming material, followed by a second stage in which the transient restoration is converted into a long-lasting dental restoration by applying further one or more curing process. Each of said first stage and second stages is independently carried out chair-side by a dentist or in a dental laboratory.

The method is preferably applied for fully structuring the dental restoration chair-side, during a single office visit. The method may also be applied for partially structuring the dental restoration in a dental laboratory, preferably commencing the first stage in which the transient dental restoration is prepared, followed by chair side conversion of the transient dental restoration into a long-lasting restoration by applying the second curing stage. The method is also suitable for fully structuring the dental restoration in a dental laboratory.

### MODES OF CARRYING OUT THE INVENTION

The present invention provides, in one aspect, dental restoration forming materials suitable for chair-side and laboratory preparation of various dental restorations during a one or more visits to the dentist's office. Dental restorations that can be prepared according to the invention include, but are not limited to crowns, fillings, inlays, onlays, veneers, bridges, splints and dentures. The dental restoration forming material is provided, according to the invention, in kits along with instructions for use thereof.

The process by which the dental restorations of the invention are formed is based on the consecutive polymerization or curing of at least two polymeric systems such that the polymerization of at least one set of monomers is first induced to obtain a plastic dental forming material. In this temporary stage, the plastic dental forming material is shaped in the form of the desired dental restoration, e.g., a dental crown, a bridge, an inlay or an onlay, using routine procedures. Then, the dental restoration is subjected to at least one more curing process under controlled conditions in order to further harden it and confer long-lasting properties to it.

The present invention differs from US 2008/0220396 in that it utilizes at least two different polymeric systems to obtain long-lasting dental restorations, one of which is the epoxy system, which is not disclosed in US 2008/0220396, whereas the two-phase light-curing composition (TPLC) of US 2008/0220396 comprises only one polymeric system.

The long-lasting dental restoration of the invention essentially comprises: (i) a polymeric matrix comprising at least two different polymeric systems; (ii) an inorganic filler; and (iii) a pigment, wherein said polymeric matrix is formed by at least two curing processes.

The curing processes are selected from, but not limited to, a self curing process, a thermo-curing process, a photo-curing process or a UV-curing process.

The term “self-curing process” as used herein refers to a polymerization process which occurs spontaneously under ambient conditions when the monomers and curing agents are brought into contact.

The term “controlled curing process” refers herein to a polymerization process which requires certain conditions in order to commence such as elevated temperatures, light or radiation at certain wavelengths, and the like. Only when these required conditions are provided, polymerization of monomers occurs in the presence of suitable curing agents. A thermo-curing process, a photo-curing process and a UV-curing process are controlled curing processes which facilitate polymerization in the presence of curing agents that are activated only at certain temperatures or at certain wave lengths.

In certain embodiments of the invention, at least one of the curing processes is a self curing process and at least one is a controlled curing process. In certain embodiments, the long-lasting dental restoration is prepared by two consecutive curing processes of which the first is a self curing process and the second is a controlled curing process.

In certain embodiments, the long-lasting restoration is formed solely by controlled curing processes. More preferably, at least one of said controlled processes is a light curing process and at least one is a UV curing process.

In a first aspect, the present invention provides a kit for the preparation of a long lasting dental restoration. In certain embodiments, the kit of the invention comprises at least the following ingredients contained in one or more containers:
(i) a first polymerizable compound;
(ii) a second polymerizable compound;
(iii) a curing agent for a first polymerization process;
(iv) a curing agent for a second polymerization process;
(v) an inorganic filler; and
(vi) a pigment;
   and optionally comprising instructions for using said ingredients.

Besides the essential ingredients listed above, the kit of the present invention may further contain at least one ingredient selected from: an accelerator for a first polymerization process, an accelerator for a second polymerization process, a polymer, a cross-linking agent, a co-reactant, an adhesion promoter such as a silane or titanate derivative, at least one additional polymerizable compound, fibers and microfibers.

When the first polymerizable compound is activated by a first curing process, a plastic dental restoration forming material is formed, such that it can be shaped while gradually hardening to obtain a partially cured dental restoration. This restoration is solid but still soft enough for deforming and carving. Only when the second polymerizable compound is activated by one or more additional curing process, said dental restoration is fully cured and hardened to obtain a long-lasting dental restoration.

The first polymerizable compound is most often an addition polymerization compound having free radically active functional groups and includes monomers, oligomers, and polymers having one or more ethylenically unsaturated groups such as acryl, methacryl, vinyl and the like. Preferably, the addition polymerization compound is a monomer comprising a mono-functional ethylenically unsaturated monomer and a multifunctional ethylenically unsaturated monomer, wherein the multifunctional monomer constitutes only 0 to 5% of said first polymerizable compound.

Di- or poly-functional monomers such as dimethacrylates, which are the major constituents of dental filling materials may constitute only a small portion of the first monomer of the invention since they polymerize into a cross-linked matrix which may start as a soft gel, however it is not a plastic gel but rather an elastic one which cannot be deformed by the dentist as it hardens. The presence of cross-linking introduces a “memory” to the gel that is not desired in accordance with the present invention.

Preferably, according to the invention, the addition mono-functional or multifunctional monomer is an acrylate or a methacrylate derivative, more preferably a methacrylate derivative such as, but not limited to, mono-, di- or poly-methacrylate including the mono-functional methyl methacrylate (MMA), isopropyl methacrylate, isobornyl-methacrylate, phenoxyethylmethacrylate, tetrahydrofurfurylmethacrylate, cyclohexyl methacrylate, isophoryl methacrylate; multifunctional methacrylates including triethyleneglycol dimethacrylate, tetraethylene glycol dimethacrylate, 1,3-propanediol dimethacrylate, trimethylolpropane trimethacrylate, 1,2,4-butanetriol trimethacrylate, 1,6-hexanediol dimethacrylate, pentaerythritol tetramethacrylate; 2,2-bis[4-(2-hydroxy-3-methacryloyloxypropoxy)phenyl]propane (Bis-GMA); 2,2-bis[4-(methacryloyloxy-ethoxy)phenyl]propane (or ethoxylated bisphenol A-dimethacrylate) (EBPADMA); urethane dimethacrylate (UDMA), diurethane dimethacrylate (DUDMA); polyurethane dimethacrylate (PUDMA); polycarbonate dimethacrylate (PCDMA); the bis-methacrylates of polyethylene glycols; copolymerizable mixtures of mathacrylated monomers; methacrylated oligomers; bis[2-(methacryloxyloxy)ethyl]phosphate; and mixtures thereof.

Methacrylates are most useful since they form relatively hard polymers and since they are relatively non-irritant and non-toxic as compared to acrylic monomers for example. The simplest methacrylate, methylmethacrylate (MMA), forms a polymer with a high glass transition temperature (Tg) of about 100° C. (referred to herein as a “hard polymer”), and is one of the preferred monomers. The glass transition temperature is a function of chain flexibility. The glass transition occurs when there is enough vibrational (thermal) energy in the system to create sufficient free-volume to permit sequences of 6-10 main-chain carbons to move together as a unit. At this point, the mechanical behavior of the polymer changes from rigid and brittle to tough and leathery, a behavior defined herein as a “plastic behavior”.

Thus, preferred mono-functional methacrylate derivatives useful for producing the dental restorations of the present invention include, but are not limited to, methacrylate MMA, isobornyl-methacrylate which forms a hard polymer with Tg of 110° C., phenoxyethylmethacrylate (Tg of 54° C.), tetrahydrofurfurylmethacrylate (Tg of 75° C.), cyclohexyl methacrylate and isophoryl methacrylate. The most preferred mono-functional methacrylate is MMA.

Other useful methacrylates include also more flexible methacrylate monomers such as ethyl, isopropyl, butyl, iso-butyl and t-butylmethacrylates; 2-ethylhexylmethacrylate, hexylmethacrylate, nonylmethacrylate, lauryl methacrylate and other linear and branched alkyl methacrylates; benzylmethacrylate, phenylmethacrylate, t-butylphenylmethacrylate, 2-methoxyethylmethacrylate, 2-ethoxyethylmethacrylate, 2-butoxyethylmethacrylate, and other listed in Sartomer's site (http://www.sartomereurope.com) and in Aldrich-Sigma (http://www.sigmaaldrich.com).

Further preferred methacrylate derivatives suitable for the purpose of the invention are those formed by the reaction of methacrylic acid with a monofunctional glycidyl ethers (epoxies) such as butyl glycidyl ether, cresyl glycidyl ether, phenyl glycidyl ether, and other epoxies such as limonene oxide. Also preferred are carbamate (urethane) adducts of hydroxyethylmethacrylate or hydroxypropylmethacrylate with mono functional isocyanates including, but not limited to, methylisocyanate, ethylisocyanate, propylisocyanate, isopropylisocyanate, butylisocyanate, isobutylisocyanate, phenylisocyanate, or an adduct of isocyanatoethylmethacrylate with an hydroxy compound such as methanol, ethanol and the like.

In certain embodiments, polymerization of the first polymeric system is carried out by a self-curing process which starts spontaneously when a first polymerizable compound, a first curing agent (also termed herein “a first initiator” or “first polymerization catalyst”), and a first accelerator are brought into contact, for example, by mixing. According to these embodiments, these three components of the self-curing system are contained in at least two separate containers or departments of the kit of the invention. For example, the polymerizable compound and accelerator are packed in one container or department and the first curing agent (the first initiator) is packed in a separate container or department.

It is to be noted that the term “catalyst” as used herein in the context of a polymerization catalyst is not to be interpreted in the chemical sense, namely a chemical that is not consumed by the reaction. “Catalyst” as referred to herein indicates a curing or an initiation agent which is present in small amounts. Some polymerization catalysts such as peroxides are not catalysts in the chemical sense because they participate in the reaction and are consumed in the course of polymerization.

In certain embodiments, the accelerator and initiator of the self-curing polymerization system form a redox-initiator pair, and induce a self-curing process once brought into contact. According to these embodiments, the curing agent is preferably selected from a peroxide such as benzoyl peroxide, an hydroxyperoxide or an azo compound (a compound bearing the functional group R—N═N—R′, in which R and R′ can be either aryl or alkyl), which can decompose in the presence of the accelerator. The first curing agent is preferably present in the composition in the amount of about 0.01 to about 4.0 wt. %, more preferably about 0.1 to about 1.0 wt. % of the total amount of ingredients.

Further according to these embodiments, the accelerator is a reducing agent selected from a tertiary amine, a transition metal salt, a transition metal complex, or an organo-metallic salt or complex, more preferably a tertiary amine. One example of a suitable tertiary amine is ethyl 4-(dimethylamino) benzoate (EDAB). Other useful tertiary amines include triethanol amine; N,N,3,5-tetramethyl aniline; 4-(dimethylamino)-phenethyl alcohol; dimethyl aminobenzoic acid ester; N,N-dimethylaniline; N,N-dimethyl-p-toluidine; dihydroxyethyl-p-toluidine; hydroxyethyl-p-toluidine and the like. The polymerization accelerator is preferably present in the composition in the amount of about 0.01 to about 2.0 wt. %, more preferably about 0.1 to about 1.0 wt. % of the total amount of ingredients.

In certain embodiments, the first polymerization system used according to the invention is the methacrylate system, employing MMA as the monomer, benzoyl peroxide as the initiator and the tertiary amine dimethyl-p-toluidine as the accelerator. This polymerization system is vastly used by dentists for the preparation of temporary dental crowns. The components of this system as well as various other suitable temporary crown and bridge materials are available from companies that supply dental products. For example, Relliance dental MFG Co. (Illinois, US) and G.C. Corporation (Tokyo, Japan).

This polymerization system is a self-curing one. Curing starts immediately after mixing the monomer and accelerator with the curing agent. In the presence of a polymethylmethacrylate polymer, a soft plastic dental forming material is formed and remains plastic for few minutes. In this time window the dental forming material can be shaped, molded and carved to obtain a dental restoration. Then the dental forming material gradually hardens and loses its elasticity. The first polymerization system usually will not form a cross-linked polymer.

The second polymerizing system, which is essential for obtaining the polymer matrix of the dental restoration of the invention, consists of a monomer, herein referred to as “a second polymerizable monomer” or “a second monomer”, and a polymerization curing agent, termed herein “a second curing agent” or “a second polymerization catalyst” or a “second initiator”. Preferably, the second monomer and the second curing agent are packed in separate containers or departments of the kit of the invention. If the second curing agent and second monomer are contained in the same container, said container must be kept refrigerated and even then the kit will have a shorter shelf-life compared to a kit in which the catalyst and monomer are separated.

The second polymerizing system, most often unlike the first one, is capable of cross-linking Cross-linked polymers are advantageous since they form matrices that are harder than the corresponding linear polymers. Therefore, curing of the second system will be induced only after the dental restoration is shaped and fitted.

The second polymerizable monomer is selected from a glycidyl ether type epoxy resin, a cycloaliphatic epoxy or epoxidized oil. For convenience, these optional second monomers are generally referred to herein as “epoxies”.

Epoxy resins are very useful for the purpose of the invention since they do not interfere with the addition polymerization of the first polymerization system and vice versa. The most preferred epoxies are the diglycidyl ether of bisphenol A (DGEBA) and the diglycidyl ether of bisphenol F, which have a lower viscosity. These products are often diluted to further reduce viscosity.

Other suitable glycidyl ether type epoxies include, but are not limited to, butyl glycidyl ether, cresyl glycidyl ether, phenyl glycidyl ether, butanediol diglycidyl ether, hexanediol diglycidyl ether, polypropyleneglycol diglycidyl ether, dimethylolcyclohexyl diglycidyl ether, or trimethylol triglycidyl ether. These epoxy resins are often used as reactive diluents, i.e., low viscosity additives that help to reduce the total viscosity but unlike inert diluents they participate in the reaction and in the buildup of the polymeric network. Low viscosity is necessary to increase the capacity of the resin to receive more inorganic filler.

Cycloaliphatic epoxies are lower viscosity and hard epoxies. Typical cycloaliphatic epoxies suitable for the purpose of the invention include, but are not limited to, 3,4-epoxycyclohexylmethyl-3,4-epoxy-cyclohexane carboxylate (EEC), bis-(3,4-epoxycyclohexyl)adipate, poly[(2-oxiranyl)-1,2-cyclohexanediol]2-ethyl-2-(hydroxymethyl)-1,3-propanediol ether, limonene dioxide, limonene monoxide, alpha pinene-oxide, and other epoxidized allyl compounds such as epoxidized diallyl phthalate and its hydrogenated and partially hydrogenated derivatives, epoxydized unsaturated cyclic compounds (substituted cyclohexene and the like) such as 3,4-epoxidyzed di(epoxidyzed allyl) tetrahydrophthalate. Suitable epoxidized oils include, but are not limited to, soybean oil, castor oil and epoxidized alpha olefins such as linear alpha olefins having 8 to 22 carbon atoms.

Curing agents suitable for the above-mentioned epoxies are chosen form a vast number of chemical groups. The cycloaliphatic epoxies are cured with either anhydrides or carboxylic acids. These curing reactions are accelerated by either a tertiary amine such as benzyldimethylamine, or a quaternary amine such as tetrabutylammonium chloride, or with a latent catalyst that is converted into its acidic form upon heating. This later catalyst is referred to herein as a “thermoinitiator”. Examples of such thermoinitiators include borontrifluoride-ethylamine and similar other BF₃-amine complexes. Tin organic compounds and other metal-organic compounds such as a compound comprising bismuth, zirconium or zinc metal atom are effective accelerators for the epoxy-anhydride reaction. The accelerators mentioned above are termed herein “accelerators for a second polymerization process” or “second polymerization accelerators”.

Most often the anhydride reaction with the epoxy also requires a co-reactant, which is a diol or a polyol. The diol or polyol reacts with the anhydride and releases a free carboxylic acid, which further reacts with the epoxy. Although the anhydrides can work without a polyol, the presence of polyol has an accelerating effect. Polyols are the more versatile components of the kit of the invention. The polyol can be compact like trimethylolpropane, di-trimethylolpropane, pentaerythrytol, di-pentaerythrytol, or it can be a long chain flexible molecule such as polypropyleneglycol, polytetramethylene glycol, polycaprolactone and other hydroxy terminated polyols having two or more hydroxy groups and a backbone structure selected from a polyether backbone, polyester backbone, urethane-based backbone, polycarbonate-based backbone, or a hydrocarbon backbone. The type of polyol, its molecular weight and the density of its functional groups may have an important effect on its final properties. Dendritic or hyperbranched polyols (e.g. Boltron® products made by Perstorp) may also be used in order to bring about a different molecular architecture and enhanced mechanical properties of the dental composition.

The glycidyl type epoxies can be cured as the cycloaliphatic epoxies but they are more often polymerized with amines. The amines can be simple monoamines but more often they are polyfunctional amines such as diethylenetriamine, triethylenetriamine, amino-ethyl-piperazine (AEP), piperidine, isophoronediamine, trimethyl-hexamethylenediamine or amine terminated polyethers (e.g. Jeffamine® series (polyoxyalkyleneamines) marketed by Huntsman). The amines can also be different adducts and reaction products including polyamides bearing excess amine groups which are commonly called poly-amino-amides or amino-amides. Any mono-functional primary amine and poly-functional primary amine may act as a curing agent and so are all poly-functional secondary amines and combinations of primary and secondary amines and their combination with tertiary amines.

Thiol groups are effective curing agents. They can be used alone or combined with tertiary amines and other mono-or poly-amines.

Glycidyl epoxies can self polymerize by a catalytic effect of either a tertiary amine or a latent catalyst such as an imidazole compound (e.g., 2-ethyl-4-methylimidazole (EMI-24)) and dicyandiamide. Tertiary amines do not react with the epoxy group but act as a catalyst for the self polymerization of the glycidyl-ether type epoxies.

Both cycloaliphatic and glycidyl epoxies can be cured by latent acidic compounds. These catalysts can either be formed by heat, as is the case with the above mentioned BF₃-amine complexes, or the acid may be generated by irradiation. Latent acidic catalysts which are activated by irradiation are termed herein “photoinitiators”. The photoinitiator is mostly a sulfonium (Ar₃S+) or iodonium (Ar₂I+) cation and the propagating species are also cations. Therefore, the curing process which is induced by irradiation (UV or light curing) is referred to as radiation cationic curing. Photoinitiators that release catalytic acids include, but are not limited to, aromatic sulfonium salts such as triarylsulfonium hexa-fluoroantimonate and triarylsulfonium hexa-fluorophosphate, diaryliodunium tetra-fluoroborate, cyclopentadiene-iron salts such as Irgacure 261(Ciba), a diazo compound, or a mixture thereof.

In one of the preferred kits of the invention, the first curing agent is a visible-light photoinitiator and the second curing agent is a UV photoinitiator.

In certain embodiments, each one of the two polymeric systems of the dental restoration is crossed linked. According to these embodiments, the two cross-linked systems form an inter-penetrating network (IPN), in which the two polymeric systems are inter-woven but not chemically linked to each other. In other certain embodiments, only one of the polymeric system is cross linked, and the two polymeric systems form a semi-IPN system.

In a particular embodiment of the invention, the dental restoration composition comprises a methylmethacrylate (MMA) polymer as the first polymeric system and an epoxy-polymer as a second polymeric system. The MMA polymer is not cross linked, however the epoxy polymeric system is preferably cross-linked. These two polymeric systems form a semi-IPN system. The semi-IPN polymers can be chemically cross-linked by the addition of a cross-linking agent. In the case of a mixture of epoxies and methacrylates, a typical cross-linker would be glycidyl methacrylate that has one functional group which is capable of reacting with a methacrylate polymeric system and another functional group which is capable of reacting with an epoxy polymeric system.

The dental restoration forming material of the invention essentially comprises a major portion of an inorganic powder as reinforcing or structural filler. The inorganic filler is a hard, natural or synthetic inorganic oxide selected from a ceramic powder such a silicon oxide (silica), quartz, metal oxides including aluminum, zirconium, titanium and iron oxides, silicates such as, but not limited to, those based on the oxides of lithium, calcium, barium, strontium, magnesium, aluminum, sodium, potassium, cerium, tin, strontium, boron, lead, and mixtures of thereof. Some of the silicates are naturally occurring (e.g., talc, kaolin) and some are not (alumina, zirconium oxide (zirconia)). Other suitable fillers are based on a metal nitride such as silicone nitride or mixtures of metal nitrides. The glasses may or may not have fluoride-releasing properties. The benefits of using fluoride-releasing glasses are well known. Such materials are capable of releasing fluoride into the oral cavity over the long term. Fluoride generally provides added protection against acid attack that can cause tooth decay.

The inorganic filler may consist of particles ranging in size from a few nanometers and up to an average of 20 microns. Typically, a mixture of a few sizes is advantageous in terms of optimal packing of the particles and gaining optimal abrasion resistance as well as desired aesthetic appearance.

The filler may comprise more than one type of a particulate material. The powdered particulate preferably comprises discrete and individual particles that are not substantially agglomerated or aggregated. The particles are characterized by having a relatively small particle size and large surface area.

In the kit of the present invention, the filler may be contained in one or more containers.

Normally the highest level of inorganic powder is desired to achieve good abrasion resistance. Furthermore, the powder particles need to bond well to the polymeric matrix. Thus, the filler is preferably treated with one or more functional compounds which bear functional groups the can react with functional groups on either one of the polymeric systems which form the polymeric matrix of the dental restoration, or both. Most preferably the functional compounds are selected from, but not limited to, silane and titanate derivatives or a mixture thereof.

Typical silane derivatives (herein also termed “silanes”) suitable for the purpose of the invention include, but are not limited to, silanes bearing a methacrylic functional group such as methacryloxypropyl trimethoxy silane; silanes bearing an epoxy group such as glycidoxy propyl trimethoxy silane or beta-(3,4-epoxycyclohexyl)ethyl trimethoxysilane; silanes comprising amino functional group such as gama-aminopropyl trimethoxy silane, gama-aminopropyl triethoxy silane or N-beta(aminoethyl)gama-aminopropyl trimethoxy silane); silanes comprising a mercapto group such as 3-mercaptopropyl trimethoxy silane; or a mixture thereof, or a mixture of one or more of the above silanes with a non-reactive or an inert silane (i.e., a silane with no functional group to connect to the organic polymer) such as phenyl trimethoxy silane and other phenyl silanes. Addition of an inert silane improves adhesion and adds hydrophobicity. The use of the hydrophobic silanes is beneficial for preventing moisture penetration into the polymer-filler interface. The inorganic filler is preferably pre-treated with the silane derivative or, alternatively, a small amount of a silane derivative may be added to the mixture of reactants and filler.

When the polymeric matrix comprises polymethylmethacrylate and epoxy polymers, which is the preferred composition according to the invention, then the particulate material will preferably be treated with a silane or titanate derivative bearing one or more functional groups capable of reacting with either the epoxy or the methacrylic groups, preferably a methacrylate silane derivative.

Incorporation of well bonded filler is mandatory for the long-lasting dental restoration of the invention as it provides the surface of the finished restoration with hardness and abrasion resistance, which are durable. The dental restoration can withstand toothbrush abrasive forces without its surface roughness increasing dramatically. Because the patient will chew and bite on the finished restoration, it is important that the restoration has good mechanical strength. In addition to their good aesthetics, the restorations made with the compositions of this invention show high fracture-resistance and compressive strength. The finished restorations will not wear away easily when subject to mastication forces.

Most preferably, the filler used according to the invention is silicate treated with an acryloxy silane and having an average particle size in the range of about 0.1 to about 10 microns. The concentration of the structural filler in the composition is generally in the range of about 20 to about 80 wt. % based on weight of the composition.

A further essential component of the dental restoration forming material of the invention is a pigment or a mixture of pigments, which is required for aesthetic reasons. Any organic and inorganic pigment is suitable for the purpose of the invention, provided it is not toxic as are some cadmium and lead compounds. The pigment must be pre-grinded into one of the components of the system, since it is unlikely to function well if added as a separate powder. The pigment is usually present in little amounts, e.g., 0.01-1% wt. % of total amount of ingredients.

The dental restoration composition of the present invention may further comprise a polymer, preferably a powdered polymer, which is a thermoplastic or a slightly cross-linked (thermosetting) polymer, and is soluble or swellable in the first polymerizable compound. The role of such a polymer is to decrease the level of shrinkage, absorb some of the exotherm of the polymerization reaction and further modify the mechanical properties of the composite. In the case where the kit of the invention comprises both a fluid and powdered components, the polymer serves as the vehicle to introduce the catalysts or accelerators. In such a case, these components shall be dissolved into the polymer either before it is being polymerized or be swelled into the powder thereof after polymerization and grinding.

In certain embodiments, particularly when the kit of the invention comprises MMA as the first monomer, the polymer is a methacrylate polymer selected from a polymethylmethacrylate (PMMA), a PMMA copolymer or a mixture thereof. The PMMA is preferably packed separately from MMA.

The kit of the invention may further contain additives to provide the dental restoration with specifically desired properties. For example, fibers and microfibers selected from carbon, graphite, zirconia, glass or ceramic fibers and microfibers. Polymeric fibers may also be used. The fibers may serve aesthetic purposes and also be used as a very effective reinforcement. They shall have a particular strong effect on tensile, flexural and impact properties.

Other additives may include fluoride-releasing agents; flavorants; pigments; fluorescent agents; opalescent agents; ultra-violet stabilizers; anti-oxidants; viscosity modifiers, and the like.

In certain embodiments, the kit of the invention will usually comprise methylmethacrylate (MMA) in the amount of 20-50% by weight as the first polymerizable compound; polymethylmethacrylate in the amount of 0-50% by weight as the polymer; a glycidyl ether type epoxy resin in the amount 5-50% by weight as the second polymerizable compound; reactive diluents and curing agents in the amount of 5-50% by weight; silicate treated with silane in the amount of 30-70% by weight, as the inorganic filler; and catalysts, accelerators, photoinitiators and pigment in a total amount of 1% to 46% by weight.

The kit provided by the invention is especially adapted for use by the dentist for chair-side preparation of dental restorations. The kit is also suitable for preparation of dental restorations in a dental laboratory by a technician.

The ingredients in the containers of the kit of the invention may be in the form of a liquid, paste or a powdered mixture. When the ingredients for preparation of a dental restoration are separated and contained within two or more containers, at least one of the containers may contain a liquid mixture of ingredients and at least one of the containers may contain a powdered mixture of ingredients and/or at least one of the containers may contain a paste-like mixture of ingredients.

In certain embodiments, the kit comprises at least two containers, one of which contains liquid ingredients and the other one contains a powdered mixture. In order to prepare a desired dental restoration, predetermined amounts of the contents of each container are first mixed to form a fluid paste or a plasticine-like dental forming material. In cases where the mixtures in the containers are all in the solid phase, a certain amount of liquid is added in order to form a paste. An example of a liquefied material that is suitable for forming a paste is poly methyl methacrylate (PMM), which also functions as a filler in the dental forming material.

When the kit of the invention comprises liquid and/or paste-like mixtures of ingredients, the dentist may mix the separately contained liquids or pates in a mixing dish or, more preferably, use a double-barrel syringe having a dispensing tip with a static mixer. The double-barrel syringe is filled with the required amounts of mixtures which are then passed through the static mixer and extruded directly into a vial or into a pre-made dental impression or a plastic matrix.

Another aspect of the present invention is the provision of a dental restoration, preferably prepared chair side. In certain embodiments, the dental restorations of the invention are long-lasting dental restorations selected from, but not limited to, dental crowns, fillings, inlays, onlays, veneers, bridges, splints and dentures, which are prepared using a kit of the invention.

The long-lasting dental restoration of the invention essentially comprises: (i) a polymeric matrix comprising at least two polymeric systems; (ii) an inorganic filler; and (iii) a pigment, and is formed by at least two curing processes of said polymeric systems. Each of said at least two curing processes is independently selected from, but not limited to, a self curing, a thermo-curing, a photo-curing or a UV-curing process.

In certain embodiments, the dental restoration is formed by two consecutive curing processes of which the first is a self-curing process and the second is a controlled curing process. Preferably, the controlled-curing process is a photo-curing process or a UV-curing process.

In certain embodiments, the dental restoration is formed by at least two consecutive controlled curing processes, the first of which is, preferably, a photo-curing process and the second is a UV-curing process. In certain embodiments, the photo-curing process is a blue-light curing process.

The polymeric systems constituting the dental restoration form an inter-penetrating-network (IPN) system or a semi-IPN system, and are most often crossed linked to each other.

In certain embodiments, the present invention provides a long-lasting dental crown. The long-lasting dental crown of the present invention, preferably prepared using a kit of the invention suitable for the preparation of a long-lasting dental crown, is fabricated either chair-side by the dentist or in a dental laboratory by a dental technician.

A long-lasting crown is used for the following non-limiting purposes: (1) protection of a weak tooth from breaking (the tooth may be weak as a result of decay or other disease); (2) holding together parts of a cracked tooth; (3) restoring a tooth that is already broken or severely worn down; (4) covering and supporting a tooth with a large filling; (5) holding a dental bridge in place; (6) covering misshaped or severely discolored tooth; or (7) covering a dental implant. The long-lasting dental crown covers a damaged tooth thereby restoring the anatomy, function, and aesthetics of part or the entire coronal portion of the natural tooth.

Common permanent crowns known in the art are usually selected from all metal, porcelain-fused-to-metal, all resin, or all ceramic crowns. Metals used in crowns include gold alloy, other alloys (for example, palladium) or a base-metal alloy (for example, nickel or chromium). Compared with other crown types, less tooth structure needs to be removed with metal crowns, and tooth wear to opposing teeth is kept to a minimum. Metal crowns withstand biting and chewing forces well and probably last the longest in terms of wear down. Also, metal crowns rarely chip or break.

Porcelain-fused-to-metal (PFM) crowns comprise vacuum fired porcelain fused on metal alloys. Dental crowns are color matched to the patient's adjacent teeth (unlike the metallic crowns). However, more wearing to the opposing teeth occurs with this crown type compared with metal or resin crowns. The crown's porcelain portion can also chip or break off. Next to all-ceramic crowns, porcelain-fused-to-metal crowns look most like normal teeth. However, sometimes the metal underlying the crown's porcelain can show through as a dark line, especially at the gum line and even more so if the gums recede.

All-ceramic or all-porcelain dental crowns are made of porcelain, resins, or dental ceramic materials. They create an extremely natural-looking appearance and are typically used on front teeth. The tooth-colored ceramic material is translucent, like the enamel of the natural teeth, and size and shade can be carefully matched. However, ceramic dental crowns are not as strong as porcelain-fused-to-metal crowns, they wear down opposing teeth a little more than metal or resin crowns, and do not function as well on back teeth, which sustain a lot of pressure from biting and chewing. Direct bonding of ceramic crown to conservative tooth preparation using low-viscosity resin cement is now common practice.

All-resin dental crowns are less expensive than other crown types. However, they wear down over time and are more prone to fractures than porcelain-fused-to-metal crowns.

In certain embodiments, long-lasting dental bridges are provided. The long-lasting dental bridges are preferably prepared using a kit of the invention suitable for the preparation of a long-lasting dental bridge, and are fabricated either chair-side by the dentist or in a dental laboratory by a dental technician. A long-lasting bridge is used for the non-limiting purposes of replacing missing tooth/teeth by joining permanently to adjacent teeth or dental implants.

In further certain embodiments, the present invention provides a long-lasting dental filling. The dental filling is preferably prepared chair side using a kit of the invention suitable for the preparation of a long-lasting dental filling. The filling is placed in the tooth if the tooth structure has enough strength and integrity to hold the filling. A filling can be placed in different areas of the tooth, for example, the occlusal and/or mesial and/or distal portions and these fillings are rated as Class I to Class VI restorations depending upon the location and condition of the tooth. A long-lasting filling is used for the non-limiting purposes of restoring the function, integrity and morphology of missing tooth structures.

In other certain embodiments, long-lasting dental inlays/onlays are provided. The long-lasting dental inlay/onlay is preferably prepared using a kit of the invention suitable for the preparation of a long-lasting dental inlay/onlay, and is fabricated either chair-side by the dentist or in a dental laboratory by a dental technician. A long-lasting inlay/onlay is used for the following non-limiting purposes: (i) repair a fractured tooth; (ii) restore a tooth when there is not enough tooth structure left; (iii) support a filling; (iv) protect a weak or badly shaped or discolored tooth; and (v) esthetic enhancement.

Veneer is a thin layer of restorative material placed over a tooth surface, either to improve the aesthetics of a tooth, e.g., a badly stained tooth that cannot be improved by teeth whitening, or to protect a damaged tooth surface (e.g., tooth that has become badly worn through excessive grinding or by carbonated drinks, as well as a tooth that has been chipped or broken). Two types of material are commonly used in a veneer, composite and porcelain. A composite veneer may be directly placed (built-up in the mouth), or indirectly fabricated by a dental technician in a dental laboratory, and later cemented to the tooth, typically using a resin cement. In contrast, a porcelain veneer may only be indirectly fabricated.

In still further certain embodiments, the present invention provides a long-lasting dental veneer. The long-lasting dental veneer is preferably prepared using a kit of the invention suitable for the preparation of a long-lasting dental veneer, and is fabricated either chair-side by the dentist or in a dental laboratory by a dental technician.

Dental dentures are removable replacements for missing teeth. They are usually made out of an acrylic resin and normally porcelain or metal will be added for additional structural support. Two types of dentures are available—complete and partial dentures. Complete dentures are used when all the teeth are missing, while partial dentures are used when some natural teeth remain.

A removable partial denture or bridge usually consists of replacement teeth attached to a pink or gum-colored plastic base, which is connected by metal framework that holds the denture in place in the mouth. Partial dentures are used when one or more natural teeth remain in the upper or lower jaw. A fixed (permanent) bridge replaces one or more teeth by placing crowns on the teeth on either side of the space and attaching artificial teeth to them. This “bridge” is then cemented into place. A fixed partial denture fills in the spaces created by missing teeth, and prevents other teeth from changing position. A precision partial denture is removable and has internal attachments rather than clasps that attach to the adjacent crowns. This provides a more natural-looking appliance.

In yet other certain embodiments, the present invention provides long-lasting dental dentures. The long-lasting dental dentures are preferably prepared using a kit of the invention suitable for the preparation of a long-lasting dental dentures, and fabricated either chair-side by the dentist or, preferably, in a dental laboratory by a dental technician.

In a further aspect, the present invention provides a method for the preparation of dental restorations. According to the method of the invention, a transient dental restoration is first formed by application of a first curing step to a dental forming material comprising two or more polymeric systems, an inorganic filler and a pigment. The first curing step may comprise one or more curing processes, which result in a transient restoration that starts as a soft plastic, plasticine-like material and gradually hardens to obtain a solid transient restoration at the end of the curing process. This transient restoration although rigid is still soft enough to be carved or processed thereby enabling further in situ processing and fitting e.g., contouring and removing excess material, and adjusting the occlusion. In a second stage of the method, the transient dental restoration is converted into a long-lasting dental restoration which is tough and non-brittle, by applying further one or more curing processes to it.

Each one of the stages of the method of the invention may, independently, be carried out chair side by the dentist or in a dental laboratory. In certain embodiments of the present invention, the first and second stages of the method are carried out, chair-side, in the dentist's office during a single visit to the office. In other certain embodiments, both steps are carried out by a dental technician in a dental laboratory. In further certain embodiments, the first stage, wherein a transient dental restoration is made, is carried out in a dental laboratory, whereas the final hardening second stage is carried out by the dentist chair side.

Once it is determined that a restoration is warranted, most often an obligatory step of the restoration process is preparation of the tooth/teeth to receive the restoration, using the techniques commonly practiced in the art. Thus, the patient is most often first anesthetized and the dentist removes any dental caries from the tooth/teeth to be restored using a dental bur or other instruments, and/or performs specific preparation work on the tooth/teeth in accordance with the type of restoration required.

In certain embodiments, the dental restoration is fabricated manually by the dentist, chair side, by directly molding and sculpturing the restoration on the prepared tooth/teeth or on a working model thereof.

In alternative embodiments, the dental restoration of the invention is prepared based on an impression or matrix of the patient's dental anatomy, which includes the tooth/teeth that will be restored, the neighboring teeth and teeth in the opposite jaw. The impression may be taken before the tooth/teeth is prepared (herein termed “a pre-operative impression”) or after preparation (herein termed a “post-operative impression”), or both before and after preparation of the tooth/teeth to be restored.

The impression may be an elastomeric impression formed using suitable impression forming materials and apparatus, for example, a plastic tray that is filled with a polyvinyl siloxane (PVS) impression material or other suitable impression materials.

For the preparation of an elastomeric impression, the dentist injects a paste-like impression material into an impression tray or matrix, and then inserts the filled tray or matrix into the patient's mouth, and the patient bites down on the paste to form the impression. The resulting impression is an accurate negative likeness of the teeth anatomy.

Alternatively, the patient's dental anatomy may be impressed digitally. A digital impression is obtained by scanning the tooth/teeth to be restored and its surrounding teeth. The digital data are collected, processed and then a three-dimensional working model of the impressed tooth/teeth is “printed” or casted.

Thus, in certain embodiments, the present invention provides a method for the preparation of a dental restoration using the kit of the invention, wherein said dental restoration is fully structured chair-side, said method comprising the steps of:

(i) optionally, taking a pre-operative impression of the tooth/teeth to be restored and adjacent teeth;

(ii) preparing the tooth/teeth to be restored;

(iii) mixing the contents of the container(s) of the kit of the invention and commencing the first curing stage to obtain a plasticine-like dental forming material;

(iv) molding the dental forming material directly on the prepared tooth/teeth and shaping it manually into the desired dental restoration; or, if a pre-operative impression has been taken in step (i) then: (a) filling the pre-operative impression with the plastic dental forming material and pressing the filled impression on the prepared tooth/teeth in the patient's mouse to cast the dental restoration; (b) releasing the casted dental restoration from the impression and placing it on the prepared tooth/teeth;

(v) adjusting the color, margins and occlusion of the restoration;

(vi) subjecting the dental restoration to further one or more controlled curing processes, thereby converting it into a long-lasting dental restoration; and

(vii) cementing the long-lasting restoration onto the prepared tooth.

In certain alternative embodiments of the invention a method for the preparation of a dental restoration using the kit of the invention is provided, wherein said dental restoration is partially structured chair-side, comprising the steps of:

(i) optionally, taking a pre-operative impression of the tooth/teeth to be restored and adjacent teeth in the patient's mouse;

(ii) preparing the tooth/teeth to be restored;

(iii) taking a post-operative impression of the patient's prepared tooth/teeth;

(iv) optionally, preparing a temporary restoration of the prepared tooth/teeth;

(v) sending the post-operative impression taken in (iii) along with the pre-operative impression, if taken in step (i), to a dental laboratory where: (a) a three-dimensional working model is printed or casted based on said impressions; (b) the contents of the container(s) of the kit of the invention are mixed and the first curing stage is initiated to obtain a plasticine-like dental forming material; (c) the plastic dental forming material is molded on the working model of the prepared tooth/teeth and shaped into the desired dental restoration; and (d) the dental restoration is sent back to the dentist;

(vi) removing the temporary restoration from the patient's mouse, if made in step (iv), and placing the dental restoration formed in (v) on the prepared tooth/teeth;

(vii) adjusting the color, margins and occlusion of the restoration;

(viii) subjecting the dental restoration to further one or more controlled curing processes, thereby converting it into a long-lasting dental restoration; and

(ix) cementing the long-lasting restoration onto the prepared tooth/teeth.

In yet further alternative embodiments, the present invention provides a method for the preparation of a dental restoration using the kit of the invention, wherein said dental restoration is fully structured in a dental laboratory, said method comprising the steps of:

(i) optionally, taking a pre-operative impression of the tooth/teeth to be restored and adjacent teeth in the patient's mouse;

(ii) preparing the tooth/teeth to be restored;

(iii) taking a post-operative impression of the patient's prepared tooth/teeth;

(iv) optionally, preparing a temporary restoration of the prepared tooth/teeth;

(v) sending the post-operative impression taken in (iii) along with the pre-operative impression, if taken in step (i), to a dental laboratory where: (a) a three-dimensional working model is printed or casted based on said impressions; (b) the contents of the container(s) of the kit of the invention are mixed and the first curing stage is initiated to obtain a plasticine-like dental forming material; (c) the plastic dental forming material is molded on the working model of the prepared tooth/teeth and shaped into the desired dental restoration; (d) the dental restoration is subjected to further one or more controlled curing processes, which convert it into a long-lasting dental restoration; and (e) the long-lasting dental restoration is sent back to the dentist;

(vi) removing the temporary restoration from the patient's mouse, if made in step (iv), and placing the long-lasting dental restoration formed in (v) on the prepared tooth/teeth;

(vii) finally adjusting occlusion of the restoration; and

(viii) cementing the long-lasting restoration onto the prepared tooth/teeth.

In certain embodiments, the long-lasting dental restorations are fully structured manually chair side during a single visit to the dentist's office.

Thus, once it is decided that a restoration is warranted and the type of restoration is determined, the dentist first prepares the tooth/teeth to be restored as commonly practiced for the kind of restoration needed. Then, the dentist prepares a dental restoration forming material using a kit of the invention suitable for the specific restoration, by mixing the ingredients contained in the one or more containers of the kit, and commencing the first curing stage of the dental forming material, as prescribed in the kit. The dental restoration forming material formed following execution of the first curing process has a paste-like texture with firmness which may vary from fluid paste to semi-solid paste such as plasticine or modeling clay. When the dental forming material is partially hardened but still plastic, it is most convenient for the dentist to mold and sculpture it manually to obtain a desired shape. The plastic dental forming material will continue to harden gradually as the first curing step progresses. At the end of the first curing process, the dental forming material loses its plasticity and hardens, but is still soft enough to sustain further carving using common dental equipment. The time window during which the dental forming material of the invention is plastic and moldable is 20 to 45 minutes.

Thus, according to the above embodiments, the dentist sculptures and molds the dental restoration forming material, while still plastic, directly on the prepared tooth/teeth in the patient's mouth, and adjusts the size and shape of the dental restoration to be compatible with neighboring teeth on both the same and opposite jaws. The manual molding and adjustment process may last 10-20 minutes, during which time the dental forming material continues to harden gradually.

The dental restoration may slightly shrink while gradually hardening, and in order to avoid its fixation to the prepared tooth before is it ready, the dentist removes the dental restoration form the patient's mouse and mounts it on again repeatedly, while carving and finally adjusting the dental restoration to optimally fit into its place. If required, the dentist may prepare a further amount of a dental formal material in a flow-like consistency and add one or more layers to the inner side of the restoration to enhance marginal and internal fitting to the prepared tooth.

When the shape and color of the dental restoration and its compatibility with surrounding teeth are satisfying, the dentist fully hardens the dental restoration by applying one or more controlled curing processes to it, preferably when the dental restoration is not in the patient's mouse. The dentist mounts the fully cured restoration on the prepared tooth/teeth and finally adjusts and polishes it, if still required. The second curing step, which completely cures the dental restoration, imparts long-lasting characteristics to it such as abrasion and fraction resistances.

Finally, the dentist cements the dental restoration onto the underling tooth/teeth using any of the suitable permanent cements applicable in dentistry.

In other certain embodiments, the long-lasting dental restoration are partially structured chair side, namely preparation of the transient dental restoration is done by a technician in a dental laboratory, whereas conversion of the temporary restoration into a long-lasting dental restoration is done by the dentist, chair side, by subjecting the restoration to one or more controlled curing processes.

Thus, according to these embodiments, once a restoration is warranted and the type of restoration is determined, the dentist first prepares the tooth/teeth to be restored as commonly practiced for the kind of restoration needed. Then, the dentist makes a post operativen impression (either elastomeric or digital) of the prepared tooth/teeth, adjacent teeth in the same and in the opposite jaws. The dentist may then make a temporary restoration of the tooth/teeth using common procedures, and release the patient. The impression is sent to a dental laboratory, where a technician first creates a working model from the impression. The technician then prepares a dental restoration forming material using a kit of the invention suitable for the specific restoration, by mixing the ingredients contained in the one or more containers of the kit, and commencing the first curing stage of the dental forming material, as prescribed in the kit. The technician fills the impression with the plastic dental restoration forming material, and mounts the impression on the working model. Before the transient dental restoration is hardened, the technician removed the dental restoration from the impression, mounts it on the working model and finally shapes it and corrects incompatibilities. The transient dental restoration is sent back to the dentist's office.

In a second visit to the dentist's office, the dentist removes the provisional dental restoration if present on the patient's prepared tooth/teeth, and mounts the transient dental restoration of the invention onto the prepared tooth/teeth, carves it, remove excess material and adjust its margins, if required, to optimally fit the dental restoration into its place. When the shape of the dental restoration and its compatibility with surrounding teeth are satisfying, the dentist fully hardens the dental restoration by applying one or more controlled curing processes to it, preferably when the dental restoration is not in the patient's mouse. The dentist mounts the fully cured restoration on the prepared tooth/teeth and finally adjusts and polishes it, if still required. The second curing step, which completely cures the dental restoration, imparts long-lasting characteristics to it such as abrasion and fraction resistances.

Finally, the dentist cements the dental restoration onto the underling tooth/teeth using any of the suitable permanent cements applicable in dentistry.

In other certain embodiments, the long-lasting dental restoration is fully structured in a dental laboratory.

According to these embodiments, once a restoration is warranted and the type of restoration is determined, the dentist first prepares the tooth/teeth to be restored as commonly practiced for the kind of restoration needed. Then, the dentist takes a post operative impression (either elastomeric or digital) of the prepared tooth/teeth and adjacent teeth in the same and in the opposite jaws. The dentist may then make a temporary restoration of the prepared tooth/teeth using common procedures, and release the patient. The impression is sent to a dental laboratory, where a technician first creates a working model based on the impression. The technician then prepares a dental restoration forming material using a kit of the invention suitable for the specific restoration, by mixing the ingredients contained in the one or more containers of the kit, and commencing the first curing stage of the dental forming material, as prescribed in the kit. The technician fills the impression with the plastic dental restoration forming material, and mounts the impression on the working model. Before the transient dental restoration hardens, the technician removes the dental restoration from the impression, mounts it on the working model and finally shapes it and corrects incompatibilities. The technician then subjects the dental restoration to one or more controlled curing processes, which confer long-lasting properties to it. In this case, the final adjustment of the restoration to the patient's mouth by the dentist is limited since the restoration is fully cured.

Thus, in a second visit to the dentist's office, the dentist removes the provisional dental restoration, if present on the patient's prepared tooth/teeth, mounts the long-lasting dental restoration of the invention onto the prepared tooth/teeth, performs minimal adjustments, checks occlusion and cements it onto its prepared site.

In still further certain embodiments, the long-lasting dental restoration is fully structured chair side during a single visit to the dentist's office, using a pre-operative impression of the tooth/teeth to be prepared.

In accordance with these certain embodiments, once a restoration is warranted and the type of restoration is determined, the dentist first takes an elastomeric pre-operative impression of the patient's dental anatomy including the tooth/teeth to be restored. The next step would be preparation of the tooth/teeth to be restored as commonly practiced for the kind of restoration needed. Then, the dentist prepares a dental restoration forming material using a kit of the invention suitable for the specific restoration, by mixing the ingredients contained in the one or more containers of the kit and commencing the first curing stage of the dental forming material, as prescribed in the kit. The dentist fills the impression with the plastic dental restoration forming material, inserts the filled impression into the patient's mouse and mounts it on the prepared tooth/teeth. The dentist releases the transient restoration form the impression, puts it back in on the prepared tooth in the patient's mouth and adjust margins and occlusion. Due to shrinkage of the transient restoration as the curing process proceeds, and in order to avoid fixation to the prepared tooth/teeth before the restoration is ready, the dentist removes the dental restoration form the prepared tooth/teeth and mounts it on again several times. If required, the dentist may prepare a further amount of a dental forming material in a flow-like consistency and add one or more layers to the inner side of the restoration to enhance marginal and internal fitting to the prepared tooth.

When the shape of the dental restoration and its compatibility with surrounding teeth are satisfying, the dentist hardens it and confers long-lasting properties to the restoration by subjecting is to controlled curing conditions, preferably when the dental restoration is not in the patient's mouse. Finally, the dentist cements the dental restoration onto the underling tooth/teeth using any of the suitable permanent cements applicable in dentistry.

In other certain embodiments, the long-lasting dental restoration are partially structured chair side using pre-operative and post operative impressions of the restored tooth/teeth, wherein preparation of the transient dental restoration, is done by a technician in a dental laboratory, and the conversion of the temporary restoration into a long-lasting dental restoration is done by the dentist, chair side, by subjecting the restoration to one or more controlled curing processes.

In accordance with these certain embodiments, once a restoration is warranted and the type of restoration is determined, the dentist first takes an elastomeric pre-operative impression of the patient's dental anatomy including the tooth/teeth to be restored. After preparation of the tooth/teeth to be restored as commonly practiced for the kind of restoration needed, the dentist takes a second impression, this time of the prepared tooth along with its neighboring teeth. Most often, the dentist makes a temporary restoration of the tooth/teeth using common procedures, and releases the patient.

The impression is sent to a dental laboratory, where a technician uses the pre-operative and post-operative impressions to create a working model of the prepared tooth/teeth. The technician then prepares a dental restoration forming material using a kit of the invention suitable for the specific restoration by mixing the ingredients contained in the one or more containers of the kit, and commencing the first curing stage of the dental forming material, as prescribed in the kit. The technician fills the pre-operative impression with the plastic dental restoration forming material, and forms a transient dental restoration using the working model as described above. The transient dental restoration is sent back to the dentist's office.

In a second visit to the dentist's office, the dentist removes the provisional dental restoration if present on the patient's prepared tooth/teeth, and mounts the transient dental restoration of the invention onto the prepared tooth/teeth, carves it, remove excess material and adjust its margins if required, to optimally fit the dental restoration into its place. When the shape of the dental restoration and its compatibility with surrounding teeth are satisfying, the dentist fully hardens the dental restoration and confers long-lasting properties to it by subjecting is to controlled curing conditions, preferably when the dental restoration is not in the patient's mouse. Finally, the dentist cements the dental restoration onto the underling tooth/teeth using any of the suitable permanent cements applicable in dentistry.

In yet other certain embodiments, the long-lasting dental restoration are fully structured in a dental laboratory using pre-operative and post operative impressions of the restored tooth/teeth taken by the dentist.

In accordance with these certain embodiments, once a restoration is warranted and the type of restoration is determined, the dentist first takes a pre-operative elastomeric impression of the patient's dental anatomy including the tooth/teeth to be restored. After preparation of the tooth/teeth to be restored as commonly practiced for the kind of restoration needed, the dentist takes a second impression, this time of the prepared tooth along with its neighboring teeth. Most often, the dentist makes a temporary restoration of the tooth/teeth using common procedures, and releases the patient.

The impression is sent to a dental laboratory, where a technician uses the pre-operative and post-operative impressions to create a working model of the prepared tooth/teeth. The technician then prepares a dental restoration forming material using a kit of the invention suitable for the specific restoration, by mixing the ingredients contained in the one or more containers of the kit, and commencing the first curing stage of the dental forming material, as prescribed in the kit. The technician fills the pre-operative impression with the plastic dental restoration forming material, mounts the impression on the working model, and before the transient dental restoration loses its plasticity, the technician removes the dental restoration from the impression, mounts it on the working model and finally adjusts and fits it using the pre-operative impression. The technician then fully cures the dental restoration by subjecting it to one or more controlled curing processes, which confer long-lasting properties to it. The transient dental restoration is sent back to the dentist's office.

In a second visit to the dentist's office, the dentist removes the provisional dental restoration, if present on the patient's prepared tooth/teeth, mounts the long-lasting dental restoration of the invention onto the prepared tooth/teeth, performs minimal adjustments, checks occlusion and cements it onto its prepared site.

In certain embodiments, the first curing step is a self curing step, which initiates once the reagents of the first polymerization system are brought into contact by mixing the contents of the one or more containers of the kit of the invention. The controlled curing processes utilized in the second curing stage, which confer long-lasting characteristics to the restoration, are selected from thermo-curing, photo-curing or UV-curing processes.

Long-lasting dental restorations which may be prepared according to any of one of the methods of the invention described above include, but are not limited to, dental crowns, fillings, inlays, onlays, veneers, bridges, splints and dentures.

In certain embodiments, a long lasting dental crown is prepared in accordance with any of the methods of the invention. Thus, for example, the dental crown may be prepared by the dentist, chair-side during a single visit to the office, wherein the dentists does not take pre-operative or post operative impressions of the tooth to be crowned and the sculpturing and modeling of the crown is done manually. Alternatively, the dentist may take a pre-operative impression of the tooth and mold the crown using this impression.

In a method of preparation of a dental crown, an obligatory step would be preparation of the tooth that will receive the crown. In order to prepare the tooth, the dentist may remove any dental caries from the tooth using a dental bur or other instruments. Then, the dentist performs “crown prep” work on the tooth by grinding it to a “core” or “stump”. Usually, from about 0.7 to 1.5 mm mass is removed from the tooth's circumference and chewing surface. A high-speed or low-speed hand piece, which is equipped with a diamond bur is used typically to grind the tooth and prepare the core.

In those embodiments wherein a dental crown is fully structured chair-side by the dentist, once the tooth that will receive the crown is prepared, the dentist uses a kit of the invention suitable for the preparation of a long-lasting dental crown and prepares a soft, plastic dental forming material from which he manually shapes and molds a crown directly on the prepared core, and then converts the thus formed transient crown into a long-lasting crown by subjecting it to one or more controlled curing processes. If a pre-operative impression has been taken, the dentist inserts the soft and plastic dental crown forming material into the impression, mounts the filled impression on the prepared tooth and casts or impresses the transient crown.

In alternative embodiments, the dentist takes a post-operative impression of the prepared tooth to be sent to a dental laboratory, where a transient or a long-lasting dental crown will be manufactured by a technician using the kit of the invention. In these cases, the dentist may temporary restore the prepared tooth in order to protect the prepared site and provide an esthetic appearance to it. This temporary restoration will be replaced with the long-lasting dental crown of the invention during a second visit to the dentist office.

A temporary crown is commonly made from a polymeric paste-like material such as an acrylic. More particularly, a polymerizable material, which is prepared from base and catalyst pastes, may be used to form the temporary crown. The base and catalysts pastes may be extruded directly onto a pre-made impression or plastic matrix to mold the polymerizable material over the prepared tooth. Then, the impression containing the molded, partially-cured material is removed from the patient's mouth, the polymerizable material is fully cured by chemical-curing, light-curing, heat-curing, or other suitable process, and the temporary crown is formed. The temporary crown is then cemented to the tooth using a temporary dental cement.

The process for denture preparation takes about three weeks to 1.5 months and several appointments. Once the dentist or prosthodontist (a dentist who specializes in the restoration and replacement of teeth) determines what type of appliance is warranted, the general steps include: (1) preparation of a series of impressions of the patient's jaws and taking measurements of how the jaws relate to one another and how much space is between them; (2) creation of models, wax forms, and/or plastic patterns in the exact shape and position of the denture to be made. The patient “tries in” this model several times and the denture is assessed for color, shape, and fit before the final denture is cast; (3) casting a final denture; and (4) performing final adjustments if necessary.

According to the method provided by the present invention, after a series of impressions are taken, a transient denture is made in the second step, using a kit of the invention suitable for preparation of denture, in the exact shape of the denture to be made. The patient “tries in” this transient denture, and the denture is assessed for color, shape, and fit. Finally, the well fitted and compatible transient denture is turned into a long-lasting denture by means of applying further curing processes to it. Regarding preparation of a long-lasting denture, the improvement provided by the method of the present invention resides in eliminating the need to form a model, a wax form, and/or a plastic pattern in the shape of the denture before the final denture is casted. Instead, a transient denture is formed, fitted and adjusted to its place, and the dentist converts it into a long-lasting denture, preferably during the same office visit.

When preparing the soft plastic mixture of a dental restoration forming material, the dentist adjusts the color of the composition to the color and hue of the patient's teeth by adding to the mixture a pigment, such as any of the ready-made pigments available. Alternatively, the outer surface of the transient restoration is colored and the color is fixated during the final hardening of the restoration.

1. A method for preparing a long-lasting dental restoration from a plastic dental restoration and utilizing at least two different polymerizable compounds, the method having at least two separate and distinct curing steps comprising:
activating a first polymerizable compound in a first curing process wherein the plastic dental restoration is formed and is then shaped and molded to yield a transient dental restoration; and,
activating at least a second polymerizable compound to harden and fully cure the transient dental restoration in at least one controlled curing process to create the long-lasting dental restoration.

2. The method according to claim 1, whereby the first curing process is a self-curing processes and the at least one controlled curing process is selected from the list including: a thermo-curing, a photo-curing, and an UV-curing process.

3. The method according to claim 2, whereby each of the at least two separate and distinct curing steps is independently performed chair-side.

4. The method according to claim 3, whereby each of the at least two separate and distinct curing steps is independently performed during a single office visit.

5. The method according to claim 2, whereby each of the at least two separate and distinct curing steps is independently performed in a dental laboratory.

6. The method according to claim 2, whereby the at least two separate and distinct curing steps are independently performed in a dental laboratory and chair-side.
